(19) United States
(12) Patent Application Publication
(10) Pub. No.: US 2024/0101602 A1
(43) Pub. Date: Mar. 28, 2024

(54) ANTIMICROBIAL PEPTIDES AND METHOD OF PREVENTING OR TREATING INFECTION BY USING THE SAME

(71) Applicants: ACADEMIA SINICA, Nankang, Taipei (TW); NATIONAL TAIWAN UNIVERSITY, Taipei (TW)

(72) Inventors: Rita P.Y. Chen, Nankang, Taipei (TW); Chiu-Ping CHENG, Nankang, Taipei (TW); Chien-Chih YANG, Nankang, Taipei (TW); Kung-Ta LEE, Nankang, Taipei (TW); Ying-Lien CHEN, Nankang, Taipei (TW); Li-Hang Hsu, Nankang, Taipei (TW); Hsin-Liang CHEN, Nankang, Taipei (TW); Sung CHEN, Nankang, Taipei (TW)

(21) Appl. No.: 18/038,592

(22) PCT Filed: Nov. 24, 2021
(86) PCT No.: PCT/US2021/060689

§ 371 Date: May 24, 2023

Related U.S. Application Data
(60) Provisional application No. 63/117,530, filed on Nov. 24, 2020.

(51) Int. Cl. C07K 7/06; A01P 1/00; A61K 45/06; A61P 31/00

(52) U.S. Cl. CPC C07K 7/06; A01P 1/00; A61K 45/06; A61P 31/00

(57) ABSTRACT

Provided is a peptide and method in preventing or treating infections caused by a wide spectrum of pathogens, including bacteria and fungus in hosts such as plants and animals. Methods of preventing or treating plant diseases and infection in animals are also provided.

### BACKGROUND

### Technical Field

The present disclosure relates to an antimicrobial peptide and the uses thereof, and in particular relates to an antimicrobial peptide comprising a spatial distribution of positively charged amino acids, and its uses as preservatives, antibiotics and disinfectants for preventing or treating infections.

### 1. Description of Related Art

The main source of food for the population of the world is agriculture. However, plants that serve as food crops are constantly under threats from a wide range of microbial pathogens, including various fungi, bacteria, viruses, and other microbial organisms. For example, plant diseases caused by Ralstonia solanacearum (Rs), Pectobacterium carotovorum subsp. carotovorum (Pcc), Botrytis cinerea (Bc), and Colletotrichum gloeosproioides (Cgl) are devastating that lead to enormous losses of crop yield. It is estimated that global crop production suffers a total of 20% to 40% yield losses due to plant pathogen infection, which poses a great threat to food security.

Currently, application of chemical pesticides and breeding of resistant crops are the most used means for disease control in plants. However, the use of chemical pesticides, including fungicides, bactericides, and nematicides, is environmentally harmful, and may be toxic or even carcinogenic. Furthermore, pathogens could also develop resistance to the pesticides and become more and more to be contained in crops. On the other hand, breeding for resistant crops also have several setbacks, including the high cost of labor and time needed for developing the resistant crops, the shortage of plant resistance genes, the easy overcome of resistance genes by pathogens, and the limited defense spectrum.

Furthermore, emergence of multi-drug resistant bacteria also poses enormous threat to human health due to abuse of conventional antibiotics. Searching for new antibiotics becomes a pressing need and common goal for scientists all over the world. In the list of antibiotic-resistant “priority pathogens” published by WHO in 2017, Acinetobacter baumannii is listed in the first tier. Multi-drug resistant Acinetobacter baumannii is the most frequent cause of nosocomial infection, particularly in intensive care units. Another threating pathogen of human health is Cryptococcus neoformans/deuterogattii species complex, a fungus that causes cryptococcosis causing worldwide infections and responsible for about 180,000 annual global deaths. Current cryptococcosis treatment includes monotherapies with 5-flucytosine or fluconazole, and combination therapies with 5-flucytosine plus amphotericin B or high-dose fluconazole. However, due to high nephrotoxicity of amphotericin B and low availability of 5-flucytosine, fluconazole is considered as a safer and less expensive antifungal drug. With increasing number of drug-resistant isolates found from the pathogens, efficacy of the drug is constantly under challenge.

There is a diversified spectrum of bacterial and fungal pathogens that invade food crops, and each of the pathogens can have an extensive range of plant hosts. Numerous bacterial and fungal pathogens also cause great risks to human health. A versatile and effective mean for pathogen control is still unavailable. Therefore, an innovative, non-toxic and non-polluting antimicrobial means is urgently needed.

### SUMMARY

The present disclosure provides a peptide with antimicrobial activity, comprising a combination of positively charged amino acid residues and hydrophobic amino acid residues, where the amino acid residues are arranged in the sequence of, or consisting essentially of, BBHBBHHBBH, wherein B represents positively charged amino acid residues and H represents hydrophobic amino acid residues. In one embodiment, the positively charged amino acid residues of the provided peptide occupy a spatial distribution of at least 150 degrees of space in the α-helical conformation of the peptide. In another embodiment, the positively charged amino acid residues occupy a spatial distribution of 180 degrees of space in the α-helical conformation of the peptide. In some embodiments, the sequence of the amino acid residues consisting essentially of BBHBBHHBBH may contain additional amino acid residues that would not adversely affect the spatial distribution of the peptide. In some embodiments, the amino acid residue consists of BBHBBHHBBH in the sequence.

In another embodiment of the present disclosure, the positively charged amino acid residues of the provided peptide form at least three positively charged clusters, and each cluster has at least two positively charged amino acid residues. In a further embodiment, the at least three positively charged clusters form a triangle shaped spatial arrangement. In another embodiment of the present disclosure, the provided peptide has at least 50% of the positively charged amino acid residues. In an embodiment, the positively charged amino acid is arginine, histidine, lysine, ornithine, diaminobutyric acid (Dab), diaminopropionic acid (Dap), 2-amino-4-guanidobutyric acid (Agb), 2-amino-3-guanidopropionic acid (Agp) or 2-amino-6-guanidohexanoic acid (Agh). In another embodiment, the hydrophobic amino acid is isoleucine, leucine, alanine, methionine, phenylalanine, tryptophan, tyrosine or valine. In a further embodiment, at least one of the amino acid residues of the peptide is a D-form amino acid.

In at least one embodiment of the present disclosure, the amino acid residues include at least one non-natural amino acid. In at least one embodiment, the least one non-natural amino acid is selected from the group consisting of ornithine, citrulline, arginosuccinic acid, thyroxine, triodothyroxine, S-adenosylmethionine, homocysteine, creatinine, ovathiol, azaserine, 3,4-dihydroxy phenylalanine (DOPA), diaminobutyric acid (Dab), diaminopropionic acid (Dap), 2-amino-4-guanidobutyric acid (Agb), 2-amino-3-guanidopropionic acid (Agp), 2-amino-6-guanidohexanoic acid (Agh) and any combination thereof.

In some embodiments, the peptide of the present application further comprises at least one modification group. In some embodiments of the present disclosure, the modification group is an N-terminal modification group, and the N-terminal modification group is a linear or branched acyl group, including a formyl group, an acetyl group, a propionyl group, a butyryl group, a pentanoyl group, a hexanoyl group, a heptanoyl group, an octanoyl group, a nonanoyl group, a decanoyl group, a dodecanoyl group, a myristyl group, a palmitoyl group, a stearyl group or any combination thereof.

In an aspect of the present disclosure, a preservative comprising the peptide as described above is provided. In another aspect, an antibiotic comprising the peptide as described above is provided. In yet another aspect, a disinfectant comprising the peptide as described above is provided.

Another aspect of the present disclosure is to provide a method of preventing or treating a plant disease comprising applying the provided peptide to the plant. In one embodiment, the plant disease is caused by phytopathogenic bacteria or fungus. In another embodiment, the phytopathogenic bacteria is Xanthomonas euvesicatoria, Xanthomonas campestris pv. Campestris, Xanthomonas oryzae pv. oryzae, Agrobacterium tumefaciens, Pectobacterium carotovorum subsp. carotovorum, Erwinia chrysanthemi, Pseudomonas syringae pv. tomato, Pseudomonas syringae pv. syringae, or Ralstonia solanacearum. In another embodiment, the phytopathogenic fungus is Colletotrichum gloeosporioides or Botrytis cinereal.

Another aspect of the present disclosure is to provide a method of preventing or treating an infection in an animal by administering the provided peptide of to the animal. In an embodiment, the infection is caused by gram-negative bacteria. In another embodiment, the infection is caused by gram-positive bacteria. In a further embodiment, the infection is caused by Escherichia coli, Acinetobacter baumannii, Klebsiella pneumonia, Pseudomonas aeruginosa, Staphylococcus aureus, Staphylococcus epidermidis, Candida albicans, Cryptococcus neoformans, Cryptococcus deuterogattii, Cryptococcus gattii, or Salmonella typhimurium.

### BRIEF DESCRIPTION OF THE DRAWINGS

The present disclosure will become more readily appreciated by reference to the following descriptions in conjunction with the accompanying drawings.

FIG. 1A shows the spatial distribution of positively charged residues of five different peptides. FIG. 1B shows the structural models of pepA, pepB, pepC, pepD, pepE, and pepD3 in an α-helical conformation. For each peptide, the left image is the wireframe model of the helical wheel, and the right image is the space-filled model from the side of the helix. Lysine residue is colored in blue. The “BBHBBHHBBH” sequence pattern is circled in pepC, pepD and pepD3.

FIG. 2A shows the circular dichroism (CD) spectra of pepA, pepB, pepC, pepD, and pepE in water. FIGS. 2B to 2F show the CD spectra of pepA, pepB, pepC, pepD, and pepE, respectively, in water, 10% trifluoroethanol (TFE), and 20% TFE. FIG. 2G shows the percentage of secondary structural content of pepA, pepB, pepC, pepD, and pepE in water, 10% TFE, and 20% TFE.

FIG. 3 shows the CD spectra of pepD in water (dotted), DOPC (gray), and POPE/POPG (black).

FIG. 4 shows the effect of antimicrobial peptides on membrane permeability of pathogenic bacteria. The data shown are from a single experiment that was repeated at least three times with similar results. Eco: E. coli; Pcc: Pectobacterium carotovorum subsp. carotovorum; Rs: Ralstonia solanacearum. A superscript “c” after the peptide name indicates that the peptide is not purified by HPLC. Bar=20 μm.

FIG. 5 shows the effect of pepD2Mc on membrane permeability of spores and mycella of pathogenic fungi Botrytis cinereal and Colletotricum gloeosporioides.

FIGS. 6A to 6D show the antimicrobial effects of pepD2Mc and pepD3Mc on detached leaves of 4-week-old tomato L390 (FIGS. 6A and 6B) and leaves of Arabidopsis Col0 plants (FIGS. 6C and 6D) after infection of Pcc. The photos shown in FIGS. 6A and 6C are from a single experiment that was repeated at least three times with similar results. In FIGS. 6B and 6D, the lesion diameter values are average±SE (n≥30). The comparisons between water- and peptide-treated samples were made using the Student's t-test (***P<0.001, ****P<0.0001).

FIGS. 7A to 7D show the antimicrobial effects of pepD2Mc and pepD3Mc on detached leaves of 4-week-old tomato L390 (FIGS. 7A and 7B) and leaves of Arabidopsis Col0 plants (FIGS. 7C and 7D) after infection of Bc. The photos shown in FIGS. 7A and 7C are from a single experiment that was repeated at least three times with similar results. In FIGS. 7B and 7D, the lesion diameter values are average±SE (n≥30). The comparisons between water- and peptide-treated samples were made using the Student's t-test (***P<0.001, ****P<0.0001).

FIGS. 8A and 8B show the stability of pepD2 (FIG. 8A) and the D-form peptide pepdD2 (FIG. 8B) in rat plasma. The upper graph shows the HPLC chromatograph profiles of the peptide incubated in the rat plasma for the indicated time, with the lower graph showing the corresponding area of the peptide peak in the HPLC chromatograph profile with different incubation time.

FIG. 9 shows the result of hemolytic assay of pepD3 and pepdD2.

FIG. 10 shows the result of cytotoxicity assay of pepD3 against HEK293 cells.

### DETAILED DESCRIPTIONS

Antimicrobial peptides (AMPs) are small defense peptides naturally produced by a wide range of organisms. Thousands of AMPs have been identified from bacteria, fungi, animals, and plants. They serve as the first-line defense in the innate immune system of these organisms against microbial intruders. AMPs possess direct microbicidal, microbiostatic or immunogenic effects. They usually consist of less than 50 amino acids and are diverse in their sequences. Due to the difference in eukaryotic and prokaryotic membrane construction, AMPs display selectivity for microbes, and thus toxic side effects against cells of higher organisms are minimized. Therefore, AMPs are a promising alternative over conventional antibiotics and pesticides to combat pathogens while ensuring human and plant health.

Although some common features, such as cationic and amphipathic, or rich in disulfide bonds, can be found among AMPs, it is difficult to predict their activities and targets from their sequences. Most of the AMP designs are based on the existing AMPs and screened by trial and error. It is known that not all the cationic and amphipathic peptides have satisfied antimicrobial activity. Moreover, many AMPs have antibacterial activity in vitro, but have no function at all in vivo.

Another problem with the agricultural application of AMPs is its cost. To save the cost of peptide synthesis by solid-phase peptide synthesis, the peptide length should be as short as possible. In addition, since orthogonal synthesis needs protected amino acids, cheaper protected amino acids are thus preferred. For example, both lysine and arginine are basic amino acids, but the price of Fmoc-Lys(Boc)-OH (Fmoc: the fluorenylmethoxycarbonyl protecting group) is about three times lower than Fmoc-Arg(Pbf)-OH.

The present disclosure provides a peptide with antimicrobial activity against pathogens including bacteria and fungi that infect plants or animals. It is found in this disclosure that a unique spatial arrangement of positively-charged residues of AMPs confers its microbial activity.

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the above examples for carrying out this disclosure without contravening its scope, for different aspects and applications.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1% to 50%, 5% to 50%, 10% to 40%, 10% to 20%, or 10% to 15%, of the number to which reference is being made.

The term “peptide” used herein refers to a short chain containing more than one amino acid monomers, in which the more than one amino acid monomers are linked to each other by amide bonds. It is to be noted that the amino acid monomers used in the peptide of the present disclosure are not limited to natural amino acids, and the amino acid sequence of the peptide can also include unnatural amino acids, compounds with similar structures, or the deficiency of amino acids.

The terms “polypeptide” and “peptide” are used interchangeably herein to refer to polymers of amino acids of any length. The polymer may be linear or branched. It may comprise modified amino acids, and may be interrupted by non-amino acids. The terms also encompass an amino acid polymer that has been modified, e.g., disulfide bond formation, glycosylation, lipidation, acetylation, phosphorylation, or any other manipulation, such as conjugation with a labeling component. The polypeptide can be isolated from natural sources, can be produced by recombinant techniques from a eukaryotic or prokaryotic host, or can be a product of synthetic procedures.

The term “non-proteinogenic amino acid” refer to those amino acids not naturally encoded or found in the genetic code of any organism. In other words, non-proteinogenic amino acids are those that are not produced by the translational machinery of an organism to assemble proteins (the proteinogenic amino acids), such as the intermediates in biosynthesis, post-translationally formed in proteins, components of bacterial cell walls, neurotransmitters and toxins. Examples of the non-proteinogenic amino acid include, but not limited to, ornithine, citrulline, arginosuccinic acid, thyroxine, triodothyroxine, S-Adenosylmethionine, homocysteine, creatinine, ovathiol, azaserine, 3,4-dihydroxy phenylalanine (DOPA), diaminobutyric acid (Dab), diaminopropionic acid (Dap), 2-amino-4-guanidobutyric acid (Agb), 2-amino-3-guanidopropionic acid (Agp) and 2-amino-6-guanidohexanoic acid (Agh).

As used herein, the term “treating” or “treatment” refers to the application or administration of one or more active agents to a subject afflicted with a disorder, a symptom or condition of the disorder, or a progression of the disorder, with the purpose to cure, heal, relieve, alleviate, alter, remedy, ameliorate, improve, or affect the disorder, the symptom or condition of the disorder, the disabilities induced by the disorder, or the progression of the disorder.

As used herein, the term “preventing” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

### EXAMPLE

Exemplary embodiments of the present disclosure are further described in the following examples, which should not be construed to limit the scope of the present disclosure.

### Example 1: Peptides with Antimicrobial Activity

Five different designs of peptides with sequences shown in Table 1 below were synthesized by the Fmoc-polyamide method on a PS3 peptide synthesizer (Protein Technologies, Inc., Arizona, USA). The C-terminus of the peptides were amidated by using Rink Amide AM resin (200 to 400 mesh, Novabiochem, Germany) as the solid support during synthesis. The N-termini of the peptides were protected by using the corresponding acids instead of an amino acid derivative in the final synthetic step. Side-chain deprotection and peptide cleavage were performed by stirring the resin in a mixture of trifluoroacetic acid/water/ethanedithiol (95/2.5/2.5, % v/v) at room temperature for 2 h. After cleavage, the resin was removed, and the peptides were precipitated by adding ten volumes of ice-cold methyl t-butyl ether (MTBE). The precipitate was collected by centrifugation at 3,000 g for 15 min at 4° C., washed twice with ice-cold MTBE, and dried under vacuum to get the crude peptides. Peptide purification was performed by reverse-phase high-performance liquid chromatography (RP-HPLC) using a C18 column (10 mm×250 mm, 10 μm, SUPELCO, Sigma-Aldrich, Germany). The eluted peptide solution was collected, lyophilized, and stored in a −30° C. freezer. Peptides were identified by matrix-assisted laser desorption ionization time-of-flight (MALDI-TOF) mass spectroscopy (AutoFlex III smartbeam, Bruker, USA).

TABLE 1
Sequence of peptides with different designs of spatial distribution of
positively charged residues

| | Positively charged | | | |
|---|---|---|---|---|
| | residues | | | SEQ |
| | | Spatial | | ID |
| Peptide | Percentage | distribution | Sequence | NO. |
| pepA | 28% | 90° | Ac-WKLLKLLLKLLKLLLKLL-NH₂ | 1 |
| pepB | 39% | 120° | Ac-WKLLKKLLKLLKKLLKLL-NH₂ | 2 |
| pepC | 50% | 150° | Ac-WKLLKKLLKKLKKLLKKL-NH₂ | 3 |
| pepD | 56% | 180° | Ac-WKKLKKLLKKLKKLLKKL-NH₂ | 4 |
| pepE | 50% | interspaced | Ac-WKLKLKLKLKLKLKLKLK-NH₂ | 5 |

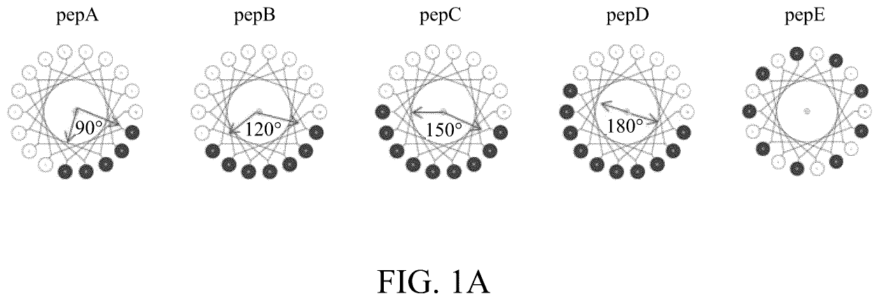

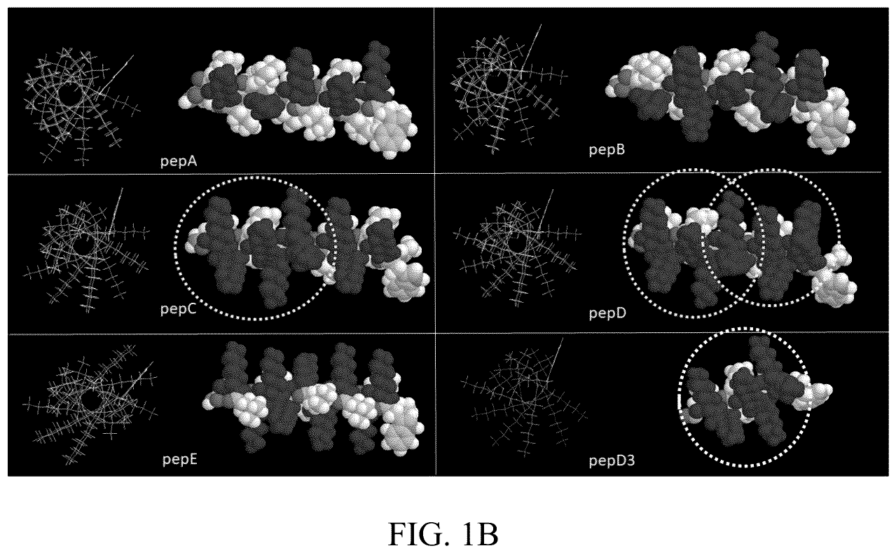

As shown in Table 1 and FIG. 1A, the five peptides pepA, pepB, pepC, pepD, and pepE are designed with different spatial distribution of positively charged residues, assuming that the peptides are in an α-helical conformation and the positively charged residues occupy 90 degrees, 120 degrees, 150 degrees, or 180 degrees of space in the α-helical conformation, or are interspaced with non-positively charged residues. The modeled image of charge distributions of the peptides in FIG. 1B show that both pepC and pepD have three positively charged clusters, with each cluster formed by two basic residues. These three positively charged clusters form a “triangle-shape” spatial arrangement.

The antimicrobial activity of the purified peptides pepA, pepB, pepC, pepD, and pepE (when a crude peptide without purification is used, it will be marked with a superscript “c”) was then tested on Xanthomonas euvesicatoria strain Xvt28, Xanthomonas campestris pv. campestris strain Xcc17, Xanthomonas oryzae pv. oryzae strain Xoo28, Agrobacterium tumefaciens strain Atu C58C1, and Escherichia coli strain DH5a. The media and growth conditions for testing the antimicrobial activity against phytopathogenic bacteria and E. coli are listed in Table 2 below.

TABLE 2
Pathogens, hosts and growth conditions used

| | | Rich/ | |
|---|---|---|---|
| | | minimal | Growth |
| Bacteria | Test host plants | media | temperatures |
| Xev | Solanaceae | 523¹/— | 28° C. |
| (Xanthomonas euvesicatoria) | (tomato, | | |
| | tobacco) | | |
| Xcc | Brassicaceae | 523/— | 28° C. |
| (Xanthomonas campestris pv. | (Arabidopsis, | | |
| campestris) | cabbage) | | |
| Xoo | Rice bacterial | 523/— | 28° C. |
| (Xanthomonas oryzae pv. | blight | | |
| oryzae) | | | |
| Atu | Crown gall | YEP²/— | 28° C. |
| (Agrobacterium tumefaciens) | | | |
| Eco | Nil | LB³/M9⁴ | 37° C. |
| (Escherichia coli) | | | |
| Pcc | Bacterial soft rot: | LB/M9 | 28° C. |
| (Pectobacterium carotovorum | Brassicaceae, | | |
| subsp. carotovorum) | Solanaceae | | |
| Ech | Orchid soft rot | LB/M9 | 28° C. |
| (Erwinia chrysanthemi) | | | |
| Pst | Tomato | KB⁵/— | 28° C. |
| (Pseudomonas syringae pv. | | | |
| tomato) | | | |
| Pss | Tobacco | KB | 28° C. |
| (Pseudomonas syringae pv. | | | |
| syringae) | | | |
| Rs | Bacterial wilt: | 523/M9 | 28° C. |
| (Ralstonia solanacearum) | Solanaceae, | | |
| | Brassicaceae, | | |
| | strawberry | | |
| Cgl | Anthracnose: | PD⁶/CD⁷ | 25~28° C. |
| (Colletotrichum | mango, | | |
| gloeosporioides) | strawberry | | |
| Bc | Grape, strawberry, | PD/CD | 25~28° C. |
| (Botrytis cinerea) | Solanaceae, | | |
| | Brassicaceae, | | |

¹523: Bacteria screening medium 523
²YEP: yeast extract peptone
³LB: Luria broth medium
⁴M9: M9 minimum medium
⁵KB: King's B medium
⁶PD: Potato dextrose medium
⁷CD: Modified Czapek-Dox (CD) minimal medium

The media were prepared according to the formula listed in Table 3 below.

TABLE 3
Formula for preparation of medium

| Medium | Component | Per Liter | |
|---|---|---|---|
| Luria broth (LB) medium | Tryptone | 10 | g |
| | Yeast extract | 5 | g |
| | NaCl | 10 | g |
| | Adjust pH to 7.0 | | |
| | Additional 15 g agar for agar medium | | |
| YEP medium | Peptone | 5 | g |
| | Yeast extract | 5 | g |
| | NaCl | 10 | g |
| | Additional 15 g agar for agar medium | | |
| 523 medium | MgSO₄•7H₂O | 0.3 | g |
| | K₂HPO₄ | 2 | g |
| | Yeast extract | 4 | g |
| | Casein hydrolysate | 8 | g |
| | Sucrose | 10 | g |
| | Additional 15 g agar for agar medium | | |
| King's B (KB) medium | Peptone | 20 | g |
| | K₂HPO₄ | 1.5 | g |
| | MgSO₄•7H₂O | 1.5 | g |
| | Glycerol | 10 | mL |
| | Additional 15 g agar for agar medium | | |
| PD medium | Potato starch | 4 | g |
| | Dextrose | 20 | g |
| | Additional 15 g agar for agar medium | | |
| M9 minimal medium | 5X M9 salts | 200 | mL |
| | 1M MgSO₄ | 2 | mL |
| | 20% Glucose | 20 | mL |
| | 1M CaCl₂ | 0.1 | mL |
| 5X M9 salts | Na₂HPO₄ | 64 | g |
| | K₂HPO₄ | 15 | g |
| | NaCl | 2.5 | g |
| | NH₄Cl | 5 | g |
| Modified Czapek-Dox (CD) | Sucrose | 20 | g |
| minimal medium | NaNO₃ | 3 | g |
| | K₂HPO₄ | 1 | g |
| | KCl | 0.5 | g |
| | MgSO₄•7H₂O | 0.5 | g |

Adjust pH to 5.0

To measure the antimicrobial activity, single colony grown on an optimal rich agar medium for each bacterium was picked and grew in 3 mL of the same rich liquid medium for 4 to 6 h. The bacterial culture was then diluted in the same medium to give a final concentration of OD₆₀₀=0.08 and then further diluted 20 folds in the same medium. In each well of a 96-well polystyrene plate, 90 μL of the diluted culture was mixed with 10 μL of the peptide to give a peptide concentration of 0, 2, 10, and 20 μg/mL (final cell concentration is 5×10⁵ CFU/mL). OD₆₀₀ was measured after incubation at an optimal temperature for 24 h. IC₅₀ was determined as the lowest peptide concentration inhibiting 50% of bacterial growth. The antimicrobial activity expressed as IC₅₀ (μg/mL) of the peptides against the pathogens is shown in Table 4 below.

TABLE 4
IC₅₀ of pepA, pepB, pepC, pepD, and pepE against five bacteria

| | Peptide (μg/mL) | | | | |
|---|---|---|---|---|---|
| Pathogen | pepA | pepB | pepC | pepD | pepE |
| Eco DH5α | — | — | 10.0 | 10.0 | 10.0 |
| Xev Xvt28 | — | 16.7 | 8.0 | 4.0 | 2.0 |
| Xcc Xcc17 | — | — | 10.0 | 4.0 | 4.0 |
| Xoo Xoo28 | — | — | 12.5 | 8.0 | 7.3 |
| Atu C58C1 | — | — | 12.5 | 10.0 | 20.0 |

“—” indicates no antimicrobial activity being detected at the highest peptide concentration (20 μg/mL) tested in this study.

To measure the antifungal activity with the media and growth conditions of Candida albicans SC5314, the Clinical and Laboratory Standards Institute (CLSI) guidelines M27-A3 were followed. In brief, 100 μL of serially diluted peptides (2-fold the final concentration) were added in a 96-well polystyrene plate (Nest Biotechnology, China) with 100 μL of Candida albicans cells. The final cell concentrations of the inoculum were 1.25×10³ CFU/mL. The final concentrations of peptides were ranged from 0.125 to 64 μg/mL. The plates were incubated for 48 h at 35° C. without shaking. As shown in Table 5 below, the minimal inhibitory concentration (MIC) was defined as the lowest concentration showing no visible growth.

TABLE 5
MIC of pepA, pepB, pepC, pepD, and pepE against
fungus Candida albicans SC5314

| | MIC |
|---|---|
| Peptide | (μg/mL) |
| pepA | — |
| pepB | — |
| pepC | 32.0 |
| pepD | 16.0 |
| pepE | — |

“—” indicates no antimicrobial activity being detected at the highest peptide concentration (64 μg/mL) tested.

From the results in Tables 4 and 5, it was shown that pepC and pepD have both antibacterial and antifungal activity against at least five plant pathogens and one human yeast pathogen, whereas pepA and pepB have relatively low antimicrobial or antifungal activity. Comparing the sequences of the peptides, the antimicrobial pepC and pepD share the same sequence pattern “KKLKKLLKKL,” which corresponds to a combination of positively charged amino acids (B) and hydrophobic amino acids (H) “BBHBBHHBBH.” This sequence pattern consisted of positively charged amino acids (B) and hydrophobic amino acids (H) confer the antimicrobial activity of pepC and pepD.

### Example 2: Structural Characteristics of Antimicrobial Peptides

The secondary structure of pepA, pepB, pepC, pepD, and pepE is investigated by circular dichroism (CD) spectroscopy. CD spectroscopy is the most used technique to explore protein/peptide secondary structure. A negative peak at around 195 nm in the CD spectrum indicates the existence of a random coil structure, whereas a positive peak at around 195 nm together with a negative peak at around 216 nm represents the presence of a β-sheet structure. α-Helix structure is characterized by the double negative peaks at 208 and 222 nm and a positive peak at around 192 nm.

CD spectroscopy was carried out by dissolving peptides in water to make a stock solution and then diluted to the solution containing different concentrations of trifluoroethanol (TFE) to a final TFE concentration of 3011M. The CD spectra between 200 and 250 nm were recorded on a J-715 CD spectrometer (JASCO, Japan). The bandwidth was set to 2 nm, and the step resolution was 0.05 nm. Each sample was scanned twice, and the average of these two measurements was smoothed by the Savitzky-Golay method to get the final CD spectrum. The CD spectra were deconvoluted by using the CD Multivariate SSE software version 2.0.1 (JASCO, Japan).

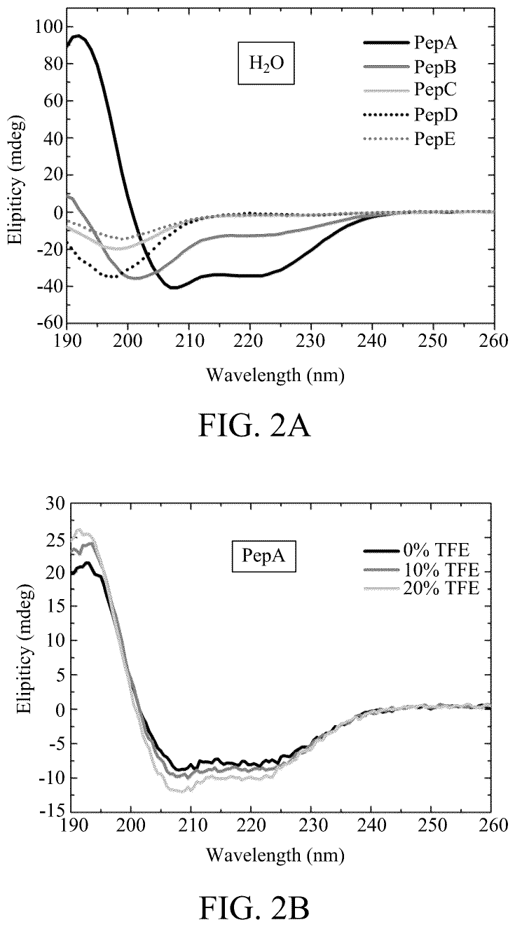

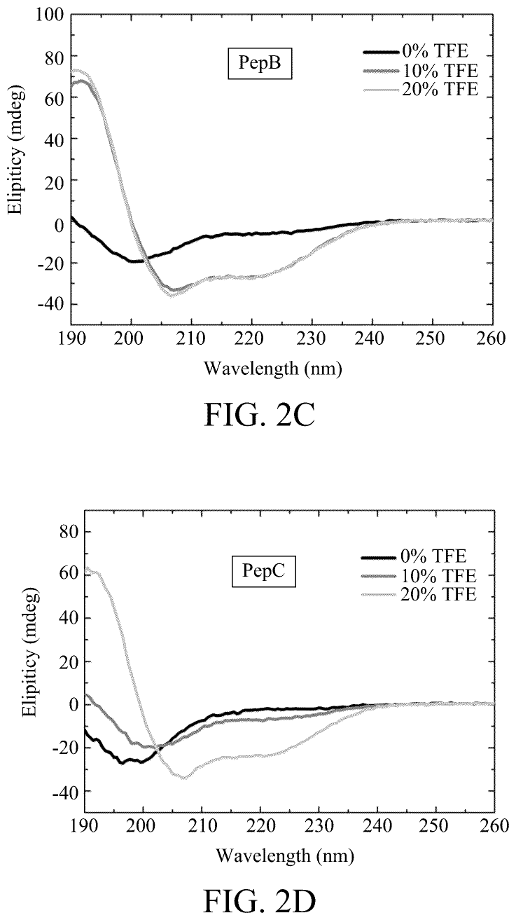

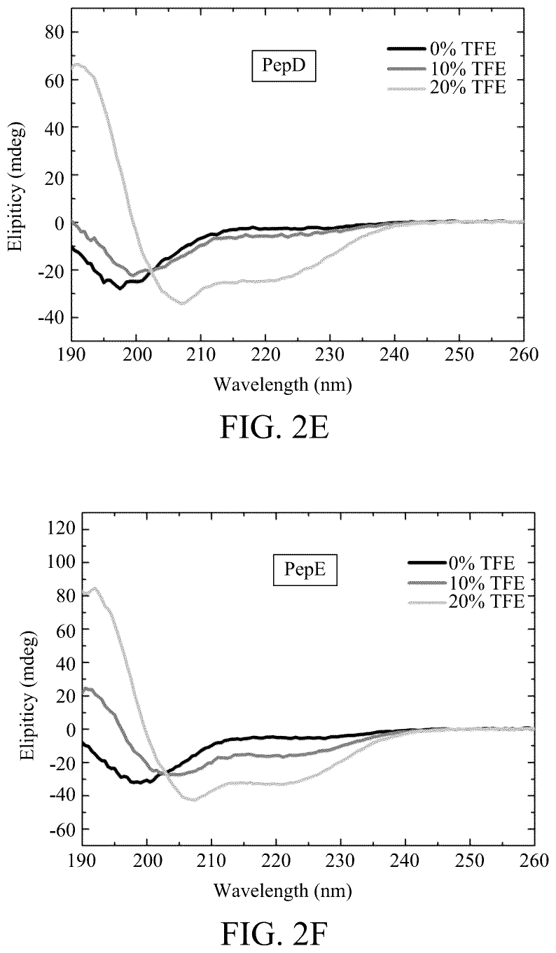

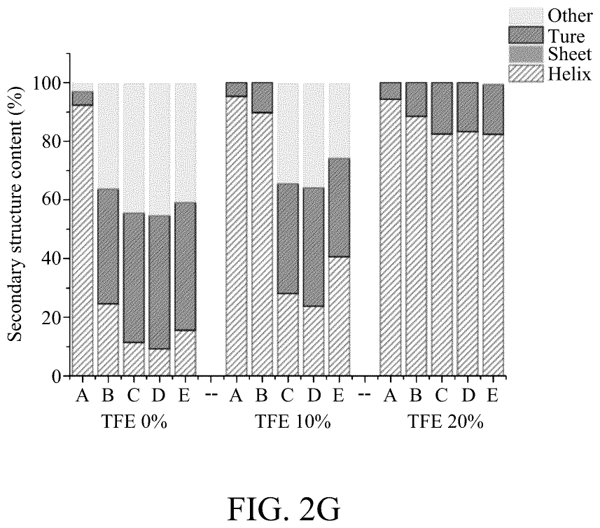

As shown in the CD spectra in FIG. 2A, in water, pepA is α-helix structure and pepB is a mixture of α-helix structure and random coil, while pepC, pepD, and pepE are random coil structures. After structural deconvolution, pepA has over 90% α-helix content; pepB has only about 25% α-helix content; and the other three peptides have lower than 20% α-helix content when they are dissolved in water (FIG. 2G). Therefore, it is shown that with increase in the ratio of positively charged residues, the structural propensity of forming α-helix structure decreases.

Trifluoroethanol (TFE) is the solvent that was commonly used to induce α-helix formation by promoting intramolecular hydrogen bond formation. The CD spectra of the peptides in water, 10% TFE, and 20% TFE were recorded and compared (FIGS. 2B to 2F). With the increase of TFE concentration, a two-state structural transition from random coil to α-helix was observed for pepB, pepC, pepD, and pepE. An isodichroic point can be found at 202 nm (FIGS. 2C to 2F). 10% TFE is enough to induce 90% α-helix formation in pepB, while 20% TFE was required for pepC, pepD, and pepE showing the CD spectra typical of α-helical structure.

Based on the structural deconvolution results of these five peptides in different TFE concentrations, the α-helix structural propensity is pepA>pepB>pepE>pepC≈pepD. Comparing with the antimicrobial activity obtained in Example 1, the α-helix structural propensity of the peptides is inversely correlated with the antimicrobial activity.

Although pepD shows a random coil structure in water, it forms an α-helical structure when interacting with certain lipids. 1,2-dioleoyl-sn-glycero-3-phosphocholine (DOPC) is commonly used to mimic mammalian cell membrane. 1-Palmitoyl-2-oleoyl-sn-glycero-3-phosphoglycerol (POPG) has a negatively charged head group. The mixture of 1-palmitoyl-2-oleoyl-sn-glycero-3-phosphoethanolamine (POPE) and POPG is commonly used to mimic the negatively charged membrane surface of bacteria.

For the CD spectra of pepD in lipids, two kinds of liposomes DOPC and POPE/POPG (1:1, w/w) were prepared. DOPC (10 mg/mL) and POPE/POPG (14 mg/mL) were dissolved in chloroform/methanol (9/1, v/v) in a glass tube individually. The solvents were evaporated under the purge of nitrogen gas to form a thin lipid film on the tube surface. The tube was placed in vacuo overnight to completely remove the organic solvent. To rehydrate the lipid film, 400 μL deionized water was added and mixed in an Intelli-Mixer (rocker mode, 60 rpm) for one hour. Then, the water/lipid mixture was frozen in liquid nitrogen and thawed at 45° C. for 5 min. After five freeze-thaw cycles, liposomes were prepared by extruding the mixture through a polycarbonate filter (with 200-nm pore size) using an Avanti Mini-Extruder (Avanti Polar Lipids, USA). Purified pepD was dissolved in water (64 μg/mL) and mixed with an equal volume of the liposome suspension for CD measurement.

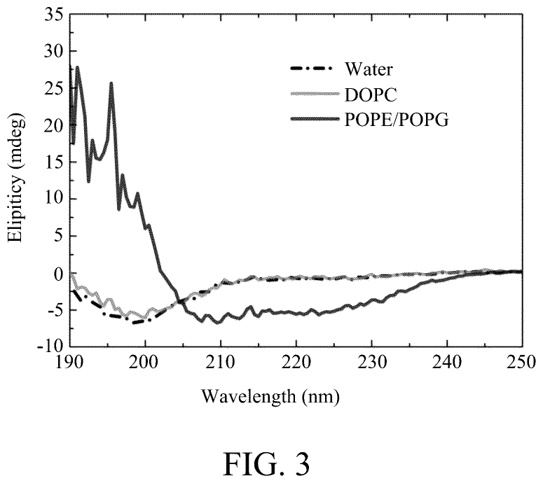

As shown in FIG. 3, pepD can be induced to populate an α-helical structure when it was mixed with the POPE/POPG mixture. This data suggests that pepD has lipid selectivity and only interacts with negatively charged lipid.

### Example 3: Antimicrobial Activity is Maintained and Improved in Shorter Peptide Variants

Two shorter peptides, pepD2 and pepD3, were synthesized and tested for their antimicrobial activity. PepD2 and pepD3 are truncated forms of pepD and are four residues and seven residues shorter than pepD, respectively, with their sequences shown in Table 6 below.

TABLE 6
Shortened peptide variant sequences of pepD

| | Carbon number | | |
|---|---|---|---|
| | at N-end | | SEQ ID |
| Peptide | modification | Sequence | NO. |
| pepD | 2 | Ac-WKKLKKLLKKLKKLLKKL-NH₂ | 4 |
| pepD2 | 2 | Ac-WKKLKKLLKKLKKL-NH₂ | 6 |
| pepD3 | 2 | Ac-WKKLKKLLKKL-NH₂ | 7 |

The effect of peptide length on the antimicrobial activity was investigated by deciding the minimal inhibitory concentration (MIC) and minimal bactericidal concentration (MBC) for each peptide against the pathogen with the media and growth conditions of phytopathogenic bacteria and E. coli as described in Table 1. Single bacterial colony grown on an optimal rich agar medium was picked, and each grew in 3 mL of the same rich liquid medium for 4 to 6 h.

For experiments carried out in the rich medium, the bacterial broth culture was then diluted in the same medium to give a final concentration of OD₆₀₀=0.08, and then further diluted 100 folds in the same medium.

For experiments carried out in the minimal medium, the bacterial cells were collected from the bacterial liquid culture by centrifugation, re-suspended in an optimal minimal medium to give a final concentration of OD₆₀₀=0.08, and then further diluted 100 folds in the same minimal medium.

Fifty microliters of serially diluted peptides (2-fold the final concentration) were added in a 96-well polystyrene plate with 50 μL of the bacterial culture. The final concentrations of peptides were 0, 2, 4, 8, 16 and 32 μg/mL. The plates were incubated at an optimal temperature without shaking. MIC was determined as the lowest concentration of peptide at which no visible bacterial growth was observed after incubation for 48 h in the rich medium or after incubation for 96 h in the minimal medium.

For bacterium-peptide mixtures that did not have obvious bacterial growth, 10 μL of the bacterium-peptide mixture was spotted on an optimal rich agar medium and incubated at an optimal temperature for 48 h. MBC was determined as the lowest peptide concentration at which no colonies formed.

The MIC and MBC results are shown in Table 7 below. The Escherichia coli and seven phytopathogenic bacteria tested include Xanthomonas euvesicatoria strain Xvt28, Xanthomonas campestris pv. Campestris strain Xcc17, Xanthomonas oryzae pv. oryzae strain Xoo28, Agrobacterium tumefaciens strain Atu C58C1, Pectobacterium carotovorum subsp. carotovorum, Pseudomonas syringae pv. tomato strain DC3000, Pseudomonas syringae pv. syringae strain B728a, and Ralstonia solanacearum strain Pss4 and strain Pss190. The result showed that shortened peptide variants of pepD maintain the antimicrobial activity and show even better antimicrobial activity than the original pepD sequence. For example, both pepD2 and pepD3 have lower MIC and MBC than pepD in Eco DH5a, Atu C58C1, Pcc, Pst DC3000 and Pss B728a, and thus better microbial activity.

TABLE 7
MIC and MBC of pepD, pepD2, and pepD3 against
E. coli and seven phytopathogenic bacteria

| | Peptide (μg/mL) | | | | | |
|---|---|---|---|---|---|---|
| | pepD | | pepD2 | | pepD3 | |
| Pathogen | MIC | MBC | MIC | MBC | MIC | MBC |
| Eco DH5α | 10.0 | 10.0 | 4.3 | 7.0 | 10.5 | 13.5 |
| Xev Xvt28 | 5.3 | 18.7 | 3.0 | 3.5 | 3.0 | 3.0 |
| Xcc Xcc17 | 2.7 | 6.7 | 1.0 | 1.0 | 2.0 | 2.0 |
| Xoo Xoo28 | 4.0 | 4.0 | 5.0 | 6.0 | 3.6 | 4.4 |
| Atu C58C1 | 4.0 | — | 1.6 | 2.2 | 2.0 | 2.4 |
| Pcc | 32.0 | — | 8.4 | 18.8 | 7.4 | 14.3 |
| Pst DC3000 | 16.0 | 24.0 | 4.8 | 8.8 | 9.3 | 17.3 |
| Pss B728a | 32.0 | — | 6.0 | 14.7 | 10.0 | 16.0 |

“—” indicates no antimicrobial activity being detected at the highest peptide concentration (32 μg/mL) tested in this study.

### Example 4: Antimicrobial Activity is Maintained and Improved in Peptide Variants with Fatty Acids Modifications

Peptides pepD2 or pepD3 were modified with fatty acids to produce myristylated (C14), palmitoylated (C16), and stearylated (C18) peptide variants and was named pepD2M or pepD3M, pepD2P, and pepD2S, respectively. The sequences of the peptide variants with fatty acids modifications are shown in Table 8 below.

TABLE 8
Peptide variants with fatty acid modifications

| | Carbon | | |
|---|---|---|---|
| | number at | | SEQ |
| | N-end | | ID |
| Peptide | modification | Sequence | NO. |
| pepD | 2 | Ac-WKKLKKLLKKLKKLLKKL-NH₂ | 4 |
| pepD2 | 2 | Ac-WKKLKKLLKKLKKL-NH₂ | 6 |
| pepD3 | 2 | Ac-WKKLKKLLKKL-NH₂ | 7 |
| pepD2M | 14 | Myristyl-WKKLKKLLKKLKKL-NH₂ | 8 |
| pepD3M | 14 | Myristyl-WKKLKKLLKKL-NH₂ | 9 |
| pepD2P | 16 | Palmitoyl-WKKLKKLLKKLKKL-NH₂ | 10 |
| pepD2S | 18 | Stearyl-WKKLKKLLKKLKKL-NH₂ | 11 |

Since the nutrients required for the growth of bacteria/fungi under the natural environments are poor, the antimicrobial activity was further tested in minimal media which better simulate the real-world scenario of pathogen infection.

Two phytopathogenic fungi Botrytis cinerea (Bc) and Colletotricum gloeosporioides were included in this test along with four other phytopathogenic bacteria. Since it is not necessary to use purified peptides in the agricultural application, crude peptides were used for this assay. The peptide SP10-5 designed by Zeitler et al. (2013) was also included in the test for comparison.

The experiments were conducted in an optimal minimal medium for each pathogen. The media and growth conditions were described in Tables 2 and 3. MIC and MBC values were determined following the procedures described above. For minimal fungicidal concentration (MFC) measurement, fungal spores (10⁶ spores/mL) suspension in sterile water were diluted in the minimal (CD) medium to give a final concentration of (2×10³ spores/mL). Fifty microliters of serially diluted peptides (2-fold the final concentration) were added in a 96-well polystyrene plate with 50 μL of the fungal spore suspension. The final concentrations of peptides were 0, 2, 4, 8, 16 and 32 μg/mL.

The plates were incubated at an optimal temperature without shaking. MIC was determined as the lowest concentration of peptide at which no visible fungal growth was observed after incubation for 72 h in the rich medium or after incubation for 96 h in the minimal medium. For fungus-peptide mixtures that did not have obvious fungal growth, 10 μL of the bacterium-peptide mixture was spotted on a PD plate and incubated at an optimal temperature for 24 to 72 h. MFC was determined as the lowest peptide concentration at which no fungal growth was observed.

The values in the table are the mean of data from at least three independent experiments. The “c” superscript indicates the crude peptide not purified by HPLC.

As shown in Table 9 below, the antimicrobial activity of the tested peptides against these different pathogens were improved under a nutrient-limited condition, which is more similar to natural environment. It is found that all the shortened peptide variants and their corresponding fatty acid-modified variants have better antimicrobial activity against the pathogens than SP10-5. In fact, the myristylated peptides were found to be more effective antimicrobial peptides than the acetylated peptides. For example, crude pepD2M and pepD3M can kill Ralstonia solanacearum, Botrytis cinerea and Colletotricum gloeosporioides, while crude pepD2 and pepD3 cannot.

TABLE 9
MIC and MBC/MFC of shortened peptide variants of
pepD and corresponding fatty acid modified variants
four phytopathogenic bacteria and two fungi

| | Pathogen | | | | | | |
|---|---|---|---|---|---|---|---|
| Peptide | | | | Rs | Rs | | Cgl |
| (μg/mL) | | Pcc | Ech | Pss4 | Pss190 | Bc | FST02 |
| pepD2^{c} | MIC | 8.0 | 16.0 | — | — | — | — |
| | MBC/ | 8.0 | 21.3 | — | — | — | — |
| | MFC | | | | | | |
| pepD2M^{c} | MIC | 4.0 | 4.0 | 8.0 | 8.0 | 12.0 | 10.0 |
| | MBC/ | 4.0 | 6.0 | 10.0 | 8.0 | 12.0 | 16.0 |
| | MFC | | | | | | |
| pepD3^{c} | MIC | 8.0 | 32.0 | — | — | — | — |
| | MBC/ | 13.3 | — | — | — | — | — |
| | MFC | | | | | | |
| pepD3M^{c} | MIC | 4.0 | 5.0 | 8.0 | 12.0 | 5.3 | 14.0 |
| | MBC/ | 4.0 | 6.0 | 12.0 | 14.0 | 6.0 | 14.0 |
| | MFC | | | | | | |
| SP10-5^{c} | MIC | 20.8 | — | — | — | — | — |
| | MBC/ | 22.4 | — | — | — | — | — |
| | MFC | | | | | | |

“—” indicates no antimicrobial activity being detected at the highest peptide concentration (32 μg/mL) tested in this study.
Pcc: Pectobacterium carotovorum subsp. Carotovorum
Ech: Erwinia chrysanthemi
Rs: Ralstonia solanacearum
Bc: Botrytis cinerea
Cgl: Colletotricum gloeosporioides

Antifungal activity of myristylated peptide pepD2M was further tested with another three fungi shown in Table 10 below, and it was found to be effective as an antimicrobial peptide. For example, it is shown that C. albicans SC5314 is susceptible to pepD2M with a MIC of 32 μg/mL and a MFC of 64 μg/mL. In addition, fluconazole resistant strain C. albicans 12-99 and echinocandin-resistant strain C. albicans 89 were also susceptible to pepD2M, suggesting that pepD2M possesses an inhibitory effect on C. albicans, including the drug-resistant strains.

TABLE 10
Antifungal activity of pepD2M

| | MIC | MFC |
|---|---|---|
| Pathogen | (μg/mL) | (μg/mL) |
| Candida albicans SC5314 | 32 | 64 |
| Candida albicans 12-99 (fluconazole-resistant) | 8 | 64 |
| Candida albicans 89 (echinocandin-resistant) | 32 | 64 |

Furthermore, the effect of the length of fatty acids used for modifications on antimicrobial activity was also investigated. As shown in Table 11 below, the palmitoylated peptide pepD2P is even more effective than the original myristylated peptide pepD2M in the optimal minimal media.

TABLE 11
MIC and MFC of fatty acid modified
peptide variants against two fungi

| | Peptide (μg/mL) | | | | | |
|---|---|---|---|---|---|---|
| | pepD2M | | pepD2P | | pepD2S | |
| Pathogen | MIC | MFC | MIC | MFC | MIC | MFC |
| Bc | 13.3 | 18.7 | 6.4 | 6.4 | — | — |
| Cgl FST02 | 14.0 | 14.0 | 8.0 | 8.0 | — | — |

“—” indicates no antimicrobial activity being detected at the highest peptide concentration (32 μg/mL) tested in this study.

### Example 5: Antimicrobial Activity is Maintained and Improved in Peptide Variants with Hydrophobic Residues Having Longer Aliphatic Chain

To study whether different hydrophobic residues may affect antimicrobial activity, peptide variants containing Leu, Ile or Val as the hydrophobic residues were synthesized and tested, with sequences shown below in Table 12.

TABLE 12
Peptide variants with substituted hydrophobic
residues

| | Carbon number | | SEQ |
|---|---|---|---|
| | at N-end | | ID |
| Peptide | modification | Sequence | NO. |
| pepD2 | 2 | Ac-WKKLKKLLKKLKKL-NH₂ | 6 |
| pepI2 | 2 | Ac-WKKIKKIIKKIKKI-NH₂ | 12 |
| pepV2 | 2 | Ac-WKKVKKVVKKVKKV-NH₂ | 13 |

Three bacteria, E. coli, Pectobacterium carotovorum subsp. Carotovorum, and Erwinia chrysanthemi, were used to compare the antimicrobial efficacy of these peptides. As shown in Table 13 below, the antimicrobial efficiency is significantly improved when Ile is used and is decreased when Val is used. Hydrophobic amino acids with longer aliphatic chain improved antimicrobial activity of the peptide.

TABLE 13
Comparison of the antimicrobial activity of the
peptides with different hydrophobic residue

| | Peptide (μg/mL) | | | | | |
|---|---|---|---|---|---|---|
| | pepD2 | | pepI2 | | pepV2 | |
| Pathogen | MIC | MBC | MIC | MBC | MIC | MBC |
| Eco DH5α | 8.0 | 8.0 | 5.3 | 5.3 | 32.0 | 32.0 |
| Pcc | 4.0 | 6.7. | 1.5 | 1.8 | — | — |
| Ech | 32.0 | 32.0 | 16.0 | 16.0 | — | — |

### Example 6: Antimicrobial Peptides Provide Membranolytic Effect of the Cell Wall

To investigate the mechanisms underlying the antimicrobial activity of the peptides, the membrane permeability of pathogens was assayed using SYTOX Green staining. This fluorescent dye is unable to enter cells to stain the nucleic acids when the cell membrane is intact, whereas it can enter cells when the cell membrane is damaged and thus the permeability is increased.

For bacterium assays, the bacteria grown in optimal rich liquid media for 16 to 20 h were collected by centrifugation at 4000×g, and then resuspended and diluted in sterile water to give a final concentration of OD₆₀₀=0.4. Five microliters of the bacterial suspension were mixed with 10 μL of SYTOX Green (2 μM) and 5 μL of the peptide (64 μg/mL), followed by incubation at room temperature in dark for 2 h.

For fungal spore assays, the spores were collected from fungal culture grown on PD plates for 7 to 10 days and removed into sterile water. The spore suspension was filtrated with Miracloth, and the infiltrate was centrifuged to collect the spores. The spores were then suspended and diluted in sterile water to give a final concentration of 10⁶ spores/mL (for Bc) or 10⁷ spores/mL (for Cgl FST02). Five microliters of the fungal spores were mixed with 10 μL of SYTOX Green (2 μM) and 5 μL of the peptide (64 μg/mL), followed by incubation at room temperature in dark for 2 h.

For fungal hyphae assays, 25 μL of the fungal spores (10⁵ spores/mL) were mixed with 75 μL of PD broth (pH=6.36 to 6.37), and then 5 μL of the spore mixture was transferred to a glass slide and kept moisture at room temperature in dark for 24 h to allow hyphal growth. Ten microliters of SYTOX Green (211M) and 5 μL of the peptide (64 μg/mL) were then added and kept moisture at room temperature in dark for 2 h. Observation of SYTOX-Green-stained cells by fluorescent microscopy was performed (the excitation light: 450 to 490 nm; the emission light: 500 nm).

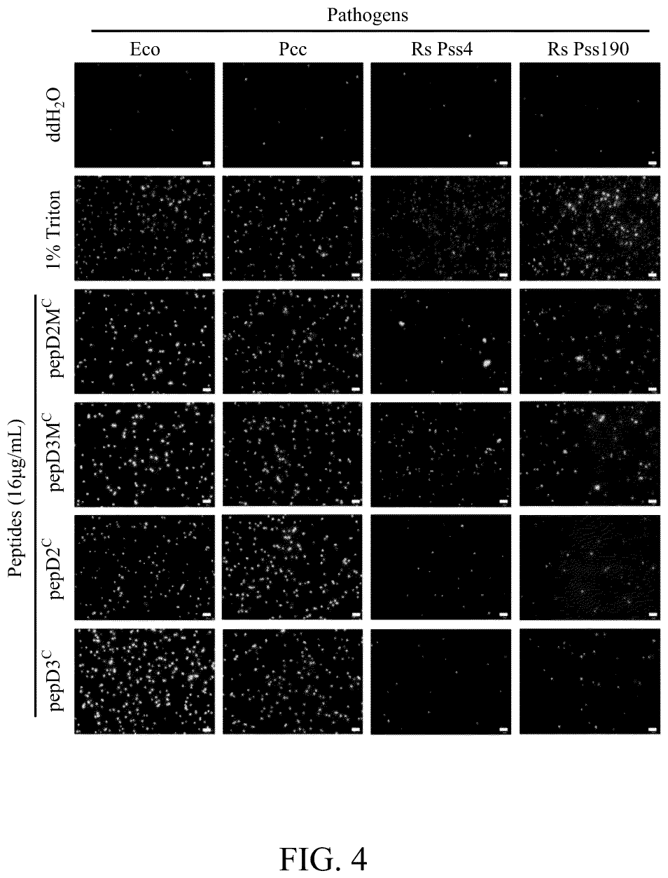

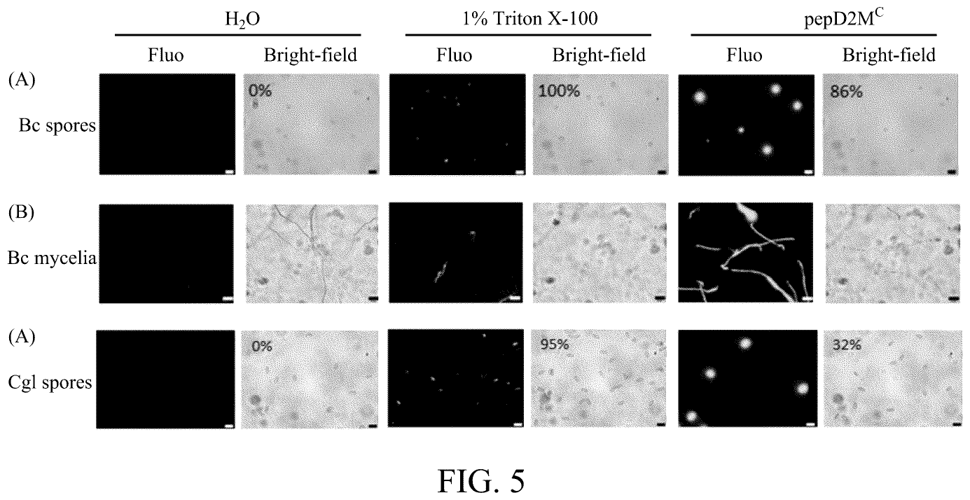

The results of FIGS. 4 and 5 showed that the peptides used in the membrane permeability test, namely pepD3^{c}, pepD2^{c}, pepD3M^{c} and pepD2M^{c}, all have significant membranolytic effect on the cell walls of bacteria, fungal spores, and fungal mycelia. These results indicate that the mechanism of the antimicrobial activity of the peptides is through membrane disruption.

### Example 7: Antimicrobial Activity in Planta

To determine in planta efficacy of selected peptides on plant disease control, the antimicrobial activity is further analyzed.

Specifically, antimicrobial effects of pepD2Mc and pepD3Mc on Arabidopsis and tomato disease responses were studied.

Four-week-old tomato L390 and Arabidopsis Col0 plants grown at their optimal temperatures (25° C. for tomato; 21° C. for Arabidopsis) with a 12h/12h light/dark cycle were used for pathogen inoculation assays.

For Pcc, the bacteria grown in LB liquid medium at 28° C. with shaking for 16 to h were collected by centrifugation at 4000×g. The collected cells were resuspended in Buffer 1 (10 mM MgSO₄, 0.01% Silwet L-77) and then serially diluted in Buffer 2 (10 mM MgSO₄) to give a final concentration of OD₆₀₀=0.002 (approximately 10⁶ CFU/mL). The Pcc suspension was mixed with the peptide (64 μg/mL) at a 3:1 ratio to give a final peptide concentration of 16 μg/mL. Detached leaves of tomato plants and leaves of intact Arabidopsis plants were wounded with a 10-μL micropipette tip and then droplet-inoculated with 10 μL of the Pcc-peptide mixture on the wounding sites. The detached tomato leaves and Arabidopsis plants were kept moisture under their optimal growth conditions. Disease symptoms were photographed 16 to 28 hours post-inoculation and diameters of lesions were measured.

For Bc inoculation, the spores were collected from fungal culture grown on PD plate for 7 to 10 days and removed into sterile water. The spore suspension was filtrated with Miracloth, and the infiltrate was centrifuged to collect the spores. The spores were then resuspended and diluted in sterile water to give a final concentration of 10⁶ spores/mL and stored at −20° C. Prior to inoculation, Bc spore suspension was diluted to give a final concentration of 10⁴ spores/mL for tomato inoculation and 10⁵ spores/mL for Arabidopsis inoculation. The diluted spore preparation was mixed with the peptide (64 μg/mL) at a 3:1 ratio to give a final peptide concentration of 16 μg/mL. Detached leaves of tomato and leaves of Arabidopsis were droplet-inoculated with 10 of the Bc-peptide mixture. The detached tomato leaves and Arabidopsis plants were kept moisture under their optimal growth conditions. Disease symptoms were photographed at 47 to 70 hours post-inoculation and diameters of lesions were measured.

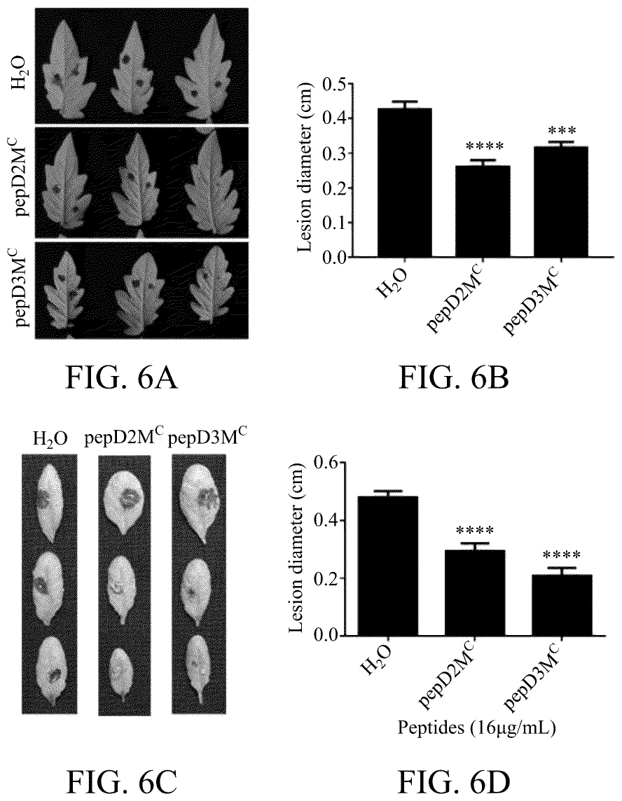

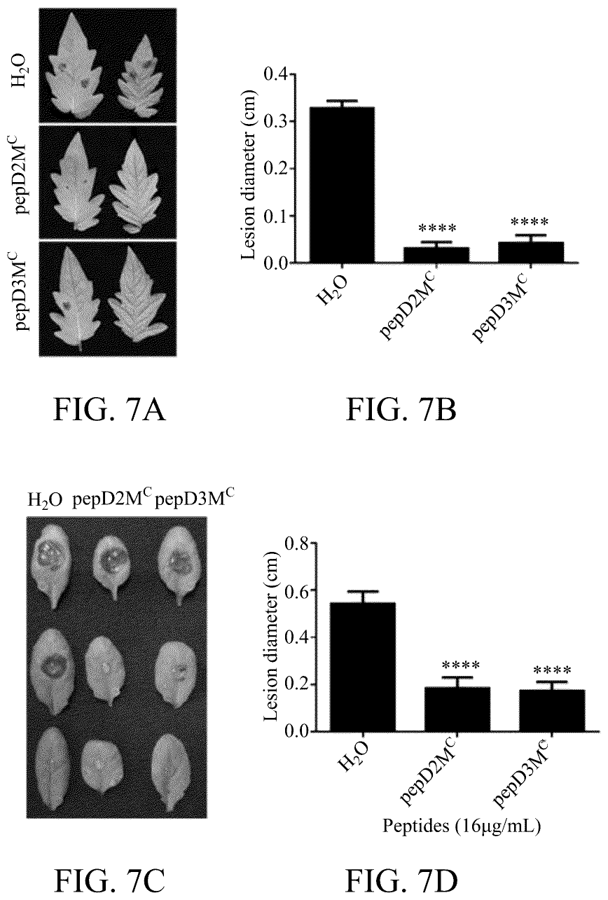

The results obtained showed that applications of pepD2Mc and pepD3Mc significantly reduced disease lesions caused by Pcc (FIGS. 6A to 6D) and Bc (FIGS. 7A to 7D).

### Example 8: Stability and Safety of Antimicrobial Peptide for Use in Animals

Peptide stability in plasma is important for killing microbes in animals. To improve peptide stability, D-form peptide variant of pepD2 (pepdD2: Ac-Wkklkkllkklkkl-NH₂ (SEQ ID No.: 14), where the small case letter represents D-form residues) was studied, using D-form Lys and D-form Leu to replace their L-form enantiomers. Similarly, D-form peptide variant of peptide D3 (pepdD3: Ac-Wkklkkllkkl-NH₂ (SEQ ID No.: 15)) was also prepared and tested for its antifungal activity.

To test the peptide stability in plasma, EDTA-treated rat whole blood was centrifuged at 4° C., 840×g for 5 minutes, and then the blood cells were removed. The supernatant was put in an Eppendorf tube and centrifuged at 4° C., 13000 rpm for 10 minutes to remove lipid (white precipitate). The supernatant was filtered through a 0.2-μm filter. The peptide was dissolved in water and filtered through a 0.2-μm filter. The peptide concentration was quantitated by UV₂₈₀ to make a stock solution of 1 mg/mL. Then, 15 μL of peptide solution was mixed with 10 μL of plasma and reacted at room temperature for 1 to 72 hours. At the indicated time, the peptide was analyzed by HPLC and a C18 column using a linear gradient of 20 to 65% solution B in 15 minutes, where the solution A is 5% acetonitrile plus 0.1% trifluoroacetic acid in water, and the solution B is 0.1% trifluoroacetic acid in acetonitrile.

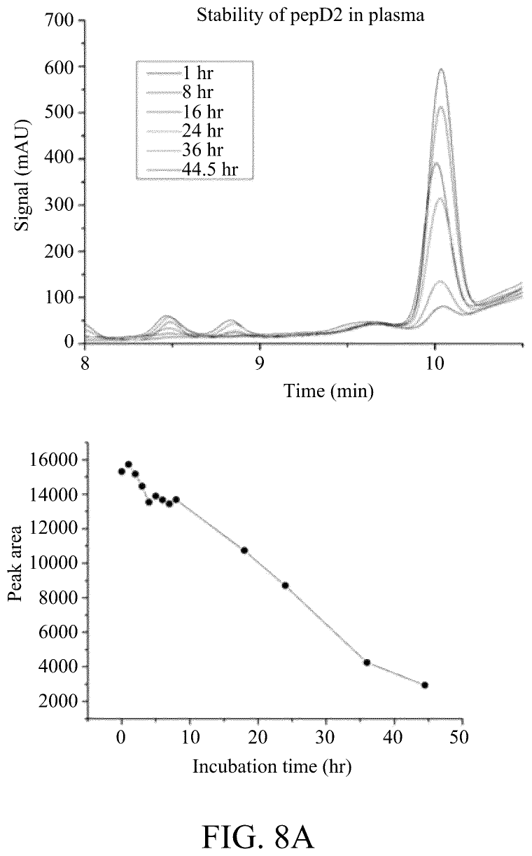

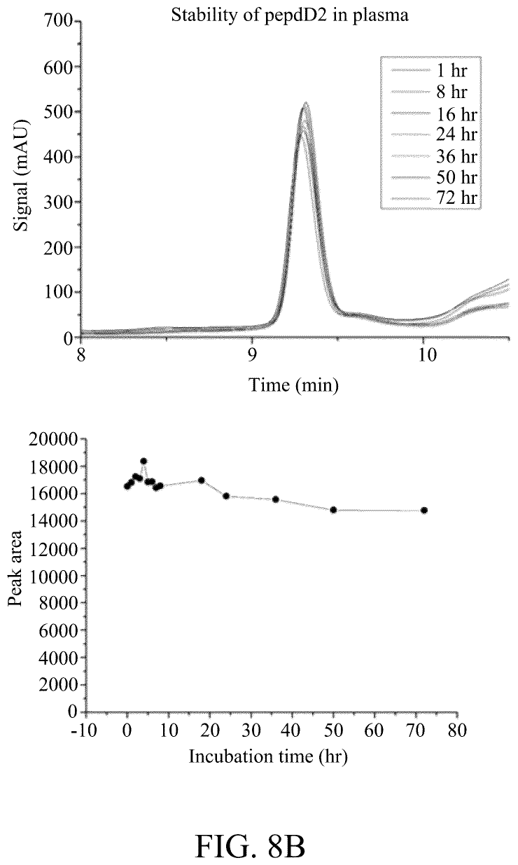

Results are shown in FIGS. 8A and 8B. It is found that the peptide using D-form residues maintains better and longer stability in plasma.

Peptide pepdD2 was used to test against more human pathogens. Table 14 below shows the MIC and MFC of pepdD2 against Cryptococcus neoformans, Cryptococcus deuterogattii, Cryptococcus gattii and Candida albicans. It is shown that pepdD2 killed all the bacteria tested, and inhibited the growth of C. neoformans H99 and C. deuterogattii R265 at the MIC of 4 and 2 μg/mL, respectively.

In addition, fluconazole resistant isolates T1 and 89-610 of C. neoformans were also susceptible to pepdD2 (MIC=2 μg/mL), suggesting that pepdD2 possesses common inhibitory effect on Cryptococcus species, as well as the drug-resistant isolates. MFCs shown in Table 14 also indicate the superior fungicidal activity of pepdD2 against Cryptococcus species.

TABLE 14
Antifungal activity of pepdD2

| | MIC | MFC |
|---|---|---|
| Pathogen | (μg/mL) | (μg/mL) |
| Cryptococcus neoformans H99 | 4 | 4 |
| Cryptococcus neoformans T1 (fluconazole-resistant) | 2 | 2 |
| Cryptococcus neoformans 89-610 (fluconazole-resistant) | 2 | 2 |
| Cryptococcus deuterogattii R265 | 2 | 2 |
| Cryptococcus gattii WM276 | 2 | 2 |
| Candida albicans SC5314 | 32 | 32 |
| Candida albicans 12-99 (fluconazole-resistant) | 16 | 32 |
| Candida albicans 89 (echinocandin-resistant) | 32 | 32 |

Antifungal activity of pepdD3 against Cryptococcus neoformans and Cryptococcus deuterogattii is shown in Table 15 below with MIC and MFC values. It is shown that C. neoformans H99 and C. deuterogattii R265 were susceptible to pepdD3 at a MIC of 8 and 4 μg/mL, respectively. In addition, fluconazole-resistant C. neoformans strains T1 and 89-610 were also shown to be susceptible to pepdD3 (MIC=4 μg/mL), suggesting that pepdD3 possesses an inhibitory effect on Cryptococcus species, including the drug-resistant strains.

TABLE 15
Antifungal activity of pepdD3

| | MIC | MFC |
|---|---|---|
| Pathogen | (μg/mL) | (μg/mL) |
| Cryptococcus neoformans H99 | 8 | 8 |
| Cryptococcus neoformans T1 (fluconazole-resistant) | 4 | 4 |
| Cryptococcus neoformans 89-610 (fluconazole-resistant) | 4 | 4 |
| Cryptococcus deuterogattii R265 | 4 | 4 |

Further, the hemolytic activity of the antimicrobial peptides was tested. EDTA-treated rat whole blood was centrifuged at 840×g for 3 minutes at 4° C. to separate blood cells and plasma. The plasma was removed, and the blood cells were washed three times with PBS having the same volume as the original blood volume by gently turning the centrifuge tube upside down. After washing, ten microliters of blood cells were diluted in PBS 2000 times to count the cells. Finally, red blood cells were diluted to 5×10⁸ cells/mL in PBS.

Peptides were dissolved in water and filtered through a 0.2-μm filter to make a stock solution. The peptide concentration was quantitated by UV₂₈₀. The peptide solution was diluted in PBS to different concentrations (5 times of tested concentration). Twenty microliters of peptide in PBS were mixed with 80 μL of red blood cell solution in a 96-well plate (V-bottom). The final peptide concentration is 16 to 256 μg/mL. In the positive control group, 20 μL of 5% Triton X-100 was added to the red blood cell solution, and the final Triton X-100 concentration was 1%. For the negative control group, 20 μL of PBS solution was added. The 96-well plate was incubated at 37° C. for 45 minutes. After centrifuging the 96-well plate at 1500×g for 5 minutes, 30 μL of the supernatant was mixed with 100 μL of deionized water in a 96-well plate (flat bottom). The concentration of heme contained in each well was measured by UV₄₀₅ using Infinite M1000 pro (Tecan). The hemolytic activity was calculated using the following formula:

Hemolytic activity=[(F−F₀)/(Fₜ−F₀)]×100%
F is UV₄₀₅ of the peptide-treated sample;
F₀ is UV₄₀₅ of sample without peptide (negative control);
Fₜ is UV₄₀₅ of the Triton-treated sample (positive control).

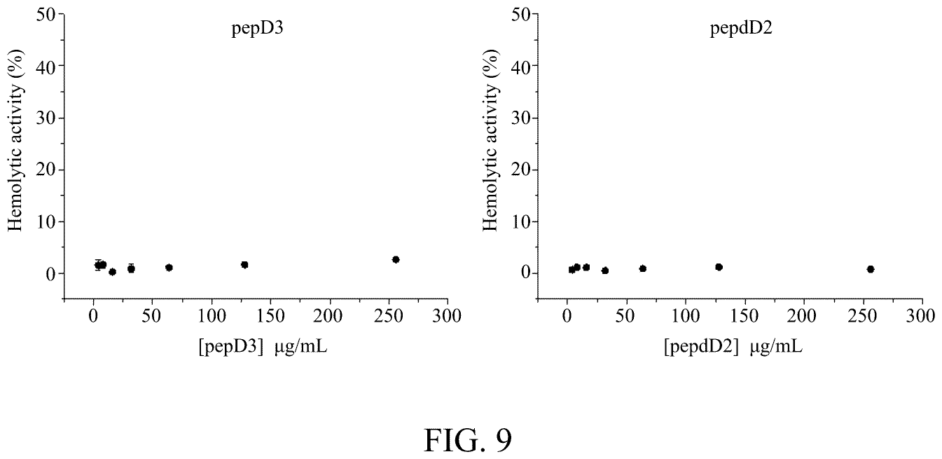

As shown in FIG. 9, both pepD3 and pepdD2 showed very little or non-hemolytic activity at concentration as high as 256m/mL.

Cell viability assay is also carried out to test for the toxicity of the antimicrobial peptides against human cells.

Cell viability assay was performed by using a 3-(4, 5-dimethylthiazol-2-yl)-5-(3-carboxymethoxyphenyl)-2-(4-sulfophenyl)-2H-tetrazolium salt (MTS) that can be reduced to purple-colored formazans by intact cells. HEK293 cells in the Dulbecco's Modified Eagle Medium (DMEM) with 10% FBS were seeded in a 96-well plate (200 μL; cell density 1.25×10⁵ cells/mL) and incubated overnight. PepD3 was dissolved in water and filtered through a 0.2-μm filter. The peptide concentration was quantitated by UV₂₈₀ to make a stock solution of 5120 μg/mL. The pepD3 stock solution was serially diluted in the serum-free DMEM. On the second day, the medium was replaced with 200 μL of peptide-containing medium (peptide concentration: 4, 8, 16, 32, 64 μg/mL) and incubated for 1h. The serum-free DMEM medium without peptide was used as negative control. Cell viability was assessed using the Cell Titer 96 Aqueous One Solution Cell Proliferation Assay (Promega), in accordance with the manufacturer's instructions. The absorbance was measured with an Infinite M1000 pro (Tecan) at a wavelength of 490 nm. Data were normalized to the negative control to obtain the percentage of cell viability.

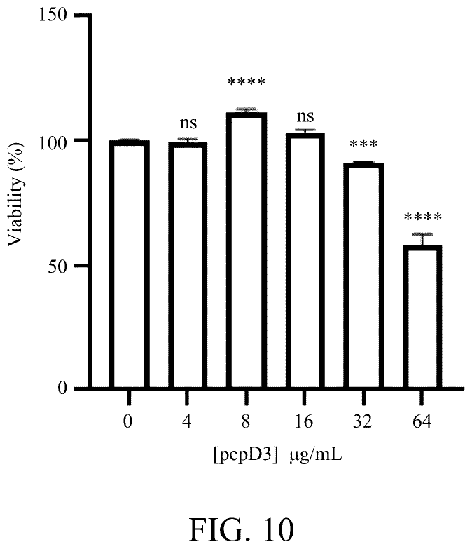

The result is shown in FIG. 10. At the concentration of 16 μg/mL, there is no significant difference on cell viability compared to the control, that is, without any peptide added. At the concentration of 32 μg/mL, the cell viability still maintains at more than 91%.

### Example 9: Efficacy of Antimicrobial Peptide Against Human Pathogens

Several human pathogens commonly cause nosocomial infections, such as Acinetobacter baumannii, Klebsiella pneumonia, Staphylococcus aureus, Staphylococcus epidermidis, Pseudomonas aeruginosa, Candida albicans, Cryptococcus neoformans, Cryptococcus deuterogattii, Cryptococcus gattii, and Salmonella typhimurium.

It was confirmed above that Leu residue in the antimicrobial “BBHBBHHBBH” sequence can be replaced by other aliphatic residues and still maintains its antimicrobial activity. To examine whether Lys can be replaced by other basic residues, two peptides, pepR2 and pepO2, were synthesized and tested.

PepR2 has the same length as pepD2, and arginine (R) was used as the positively charged residue in place of lysine (pepR2: Ac-WRRLRRLLRRLRRL-NH₂ (SEQ ID NO.: 16)). PepO2 has the same length as pepD2, and ornithine, the amino acid with one methylene group shorter than lysine, was adopted as the positively charged residue (PepO2: Ac-WOOLOOLLOOLOOL-NH₂ (SEQ ID NO.: 17).

In addition, peptide variants pepV2M (Myristyl-WKKVKKVVKKVKKV-NH₂ (SEQ ID NO.: 18)) and pepI2M (Myristyl-WKKIKKIIKKIKKI-NH₂ (SEQ ID NO.: 19)) are the pepV2 and pepI2 peptides with substituted hydrophobic residues in Example 5 that are further modified with fatty acids, e.g., myristylated, and tested for antimicrobial activity.

More peptide variants of pepD3 were provided and tested as well. Instead of using only one kind of hydrophobic aliphatic amino acid in the peptides, peptide pepD3(V)M was synthesized and myristylated, in which leucine (L) and valine (V) have equal occupancy (pepD3(V)M: Myristyl-WKKVKKLVKKL-NH₂ (SEQ ID NO.: 20)), in addition to pepD3 peptide variants having substitution of the basic residue lysine with histidine (pepD3-5H: Ac-WKKLHKLLKKL-NH₂ (SEQ ID NO.: 24)) or arginine (pepD3-5R: Ac-WKKLRKLLKKL-NH₂ (SEQ ID NO.: 25)), and pepD3 peptide variants having substitution of the hydrophobic residue leucine with alanine (pepD3-7A: Ac-WKKLKKALKKL-NH₂ (SEQ ID NO.: 26)), phenylalanine (pepD3-7F: Ac-WKKLKKFLKKL-NH₂ (SEQ ID NO.: 27)), isoleucine (pepD3-7I: Ac-WKKLKKILKKL-NH₂ (SEQ ID NO.: 28)) and methionine (pepD3-7M: Ac-WKKLKKMLKKL-NH₂ (SEQ ID NO.: 29)).

Additional peptide variants of pepD3 with fatty acid modifications such as octanoylated (C8), hexanoylated (C6), and butyrylated (C4) peptide variants were prepared and named pepD3O (Octanoyl-WKKLKKLLKKL-NH₂ (SEQ ID NO.: 21)), pepD3H (Hexanoyl-WKKLKKLLKKL-NH₂ (SEQ ID NO.: 22)) and pepD3B (Butyryl-WKKLKKLLKKL-NH₂ (SEQ ID NO.: 23)), respectively.

The sequences of the peptide variants mentioned above are listed in Table 16 below.

TABLE 16
Peptide variants of pepD2 and pepD3 tested for antimicrobial activity
against human pathogen

| | Carbon | | |
|---|---|---|---|
| | number at | | |
| | N-end | | SEQ ID |
| Peptide | modification | Sequence | NO. |
| pepD3 | 2 | Ac-WKKLKKLLKKL-NH₂ | 7 |
| pepdD2 | 2 | Ac-Wkklkkllkklkkl-NH₂ | 14 |
| pepR2 | 2 | Ac-WRRLRRLLRRLRRL-NH₂ | 16 |
| pepO2 | 2 | Ac-WOOLOOLLOOLOOL-NH₂ | 17 |
| pepV2M | 14 | Myristyl-WKKVKKVVKKVKKV-NH₂ | 18 |
| pepI2M | 14 | Myristyl-WKKIKKIIKKIKKI-NH₂ | 19 |
| pepD3(V)M | 14 | Myristyl-WKKVKKLVKKL-NH₂ | 20 |
| pepD30 | 8 | Octanoyl-WKKLKKLLKKL-NH₂ | 21 |
| pepD3H | 6 | Hexanoyl-WKKLKKLLKKL-NH₂ | 22 |
| pepD3B | 4 | Butyryl-WKKLKKLLKKL-NH₂ | 23 |
| pepD3-5H | 2 | Ac-WKKLHKLLKKL-NH₂ | 24 |
| pepD3-5R | 2 | Ac-WKKLRKLLKKL-NH₂ | 25 |
| pepD3-7A | 2 | Ac-WKKLKKALKKL-NH₂ | 26 |
| pepD3-7F | 2 | Ac-WKKLKKFLKKL-NH₂ | 27 |
| pepD3-7I | 2 | Ac-WKKLKKILKKL-NH₂ | 28 |
| pepD3-7M | 2 | Ac-WKKLKKMLKKL-NH₂ | 29 |

The efficacies of antimicrobial peptides against human pathogens were confirmed by determining the MIC and MBC.

Following the CLSI guidelines M07-A11, single bacterial colony grown on Müller-Hinton agar (MHA) plates was picked and inoculated in 4 mL of Müller-Hinton broth (MHB) for 4 to 6 h. The bacterial broth was diluted in the same medium to give a cell density (1-2×10⁸ CFU/mL) (e.g., OD₆₀₀=0.08˜0.12 for Staphylococcus aureus; OD₆₀₀=0.38˜0.4 for Acinetobacter baumannii). Then, the broth was diluted 20 folds.

Peptides to be tested were dissolved in water to make a stock solution. Then, the peptide solution was serially diluted in MHB to make the peptide concentration 1, 2, 4, 8, 16, and 32 μg/mL. One hundred microliters of serially diluted peptide and 10 μL of the bacterial culture were mixed in a 96-well polystyrene plate. Positive control is a mixture of 100 μL of MHB and 10 μL of the bacterial culture. Negative control is 110 μL of MHB. The plates were incubated at 37° C. without shaking. MIC was determined as the lowest concentration of peptide at which no visible bacterial growth was observed after incubation for 20 h in the MHB. The bacterium-peptide mixtures (without visible growth, and the mixture containing 2-fold lower peptide concentration than the mixture without growth), positive control, and negative control (3 μL each) were spotted on an MHA plate and incubated at 37° C. for 24 h. MBC was determined as the lowest peptide concentration at which no colony was formed. The antimicrobial activities against Acinetobacter baumannii of the peptide variants with different hydrophobic aliphatic residues or positively charged residues were shown in Table 17 below. The data were obtained from two independent experiments (each contains three replicates). The experiments were conducted in MHB medium. The concentration of 1, 2, 4, 8, 16, and 32 μg/mL of peptides were added in the bacterial culture.

The results showed that, although peptides comprising different hydrophobic aliphatic residues and different positively charged residues can have varied bactericidal activity, all the peptides having the “BBHBBHHBBH” sequence pattern are able to kill Acinetobacter baumannii. In other words, lysine can be substituted by ornithine or arginine. Various fatty acid modifications including pepD30, pepD3H, pepD3B maintain the MIC and MBC of pepD3. It is shown that pepI2 is the most effective peptide against Acinetobacter baumannii.

TABLE 17
MIC and MBC of peptides against
Acinetobacter baumannii ATCC 17978

| | MIC | MBC |
|---|---|---|
| Peptide | (μg/mL) | (μg/mL) |
| pepD2 | 8 | 8 |
| pepD3 | 8 | 8 |
| pepI2 | 4 | 4 |
| pepV2 | 16 | 32 |
| pepR2 | 8 | 8 |
| pepO2 | 8 | 8 |
| pepV2M | 16 | 16 |
| pepI2M | 32 | 32 |
| pepD3(V)M | 16 | 16 |
| pepD3O | 8 | 8 |
| pepD3H | 8 | 8 |
| pepD3B | 8 | 8 |
| pepdD2 | 8 | 8 |

Tables 18 to 22 show the MIC and MBC of the peptide variants against more human pathogens, including Klebsiella pneumonia NTUH-K2044, Staphylococcus aureus BCRC 10777, Staphylococcus epidermidis ATCC 14990, Pseudomonas aeruginosa ATCC 9027 and Salmonella typhimurium NCHU 15721.

Peptide variants listed in Table 18, either with fatty acid modification or substitution of basic or hydrophobic residues, are shown to retain their antimicrobial activity with an MIC or MBC ranging from 4 μg/mL to 32 μg/mL, with some of them showing even better antimicrobial activity than the unmodified or unsubstituted peptides pepD2 or pepD3.

TABLE 18
MIC and MBC of peptides against
Klebsiella pneumonia NTUH-K2044

| | MIC | MBC |
|---|---|---|
| Peptide | (μg/mL) | (μg/mL) |
| pepD2 | 16 | 16 |
| pepD3 | 16 | 16 |
| pepI2 | 16 | 32 |
| pepR2 | 32 | 32 |
| pepO2 | 16 | 32 |
| pepD3O | 16 | 16 |
| pepD3H | 16 | 16 |
| pepD3B | 16 | 16 |
| pepdD2 | 32 | 32 |

TABLE 19
MIC and MBC of peptides against
Staphylococcus aureus BCRC 10777

| | MIC | MBC |
|---|---|---|
| Peptide | (μg/mL) | (μg/mL) |
| pepD2 | 16 | 16 |
| pepD3 | 16 | 32 |
| pepI2 | 32 | 32 |
| pepR2 | 16 | 16 |
| pepO2 | 32 | 32 |
| pepV2M | 8 | 16 |
| pepD3(V)M | 16 | 16 |
| pepD3O | 8 | 8 |
| pepD3H | 8 | 8 |
| pepD3B | 16 | 16 |
| pepdD2 | 32 | 32 |

TABLE 20
MIC and MBC of peptides against
Staphylococcus epidermidis ATCC 14990

| | MIC | MBC |
|---|---|---|
| Peptide | (μg/mL) | (μg/mL) |
| pepD2 | 16 | 32 |
| pepD3 | 16 | 32 |
| pepI2 | 16 | 32 |
| pepR2 | 16 | 16 |
| pepO2 | 32 | 32 |
| pepV2M | 16 | 16 |
| pepD3(V)M | 16 | 16 |
| pepD3O | 8 | 8 |
| pepD3H | 16 | 16 |
| pepD3B | 16 | 16 |
| pepdD2 | 16 | 32 |

TABLE 21
MIC and MBC of peptides against
Pseudomonas aeruginosa ATCC 9027

| | MIC | MBC |
|---|---|---|
| Peptide | (μg/mL) | (μg/mL) |
| pepD3H | 4 | 8 |
| pepdD2 | 4 | 4 |
| pepI2 | 4 | 4 |
| pepD3 | 4 | 8 |
| pepD3-5H | 4 | 8 |
| pepD3-5R | 4 | 8 |
| pepD3-7A | 4 | 8 |
| pepD3-7F | 4 | 8 |
| pepD3-7I | 4 | 4 |
| pepD3-7M | 4 | 8 |

TABLE 22
MIC and MBC of peptides against
Salmonella typhimurium NCHU 15721

| | MIC | MBC |
|---|---|---|
| Peptide | (μg/mL) | (μg/mL) |
| pepD3H | 16 | 32 |
| pepdD2 | 16 | 16 |
| pepI2 | 8 | 16 |

While some of the embodiments of the present disclosure have been described in detail in the above, it is, however, possible for those of ordinary skill in the art to make various modifications and changes to the embodiments shown without substantially departing from the teaching and advantages of the present disclosure. Such modifications and changes are encompassed in the scope of the present disclosure as set forth in the appended claims.

1. A peptide with antimicrobial activity comprising a combination of positively charged amino acid residues and hydrophobic amino acid residues in the sequence of BBHBBHHBBH, wherein B represents positively charged amino acid residues and H represents hydrophobic amino acid residues.

2. The peptide according to claim 1, wherein the positively charged amino acid residues occupy a spatial distribution of at least 150 degrees of space in α-helical conformation of the peptide.

3. The peptide according to claim 2, wherein the positively charged amino acid residues form at least three positively charged clusters each having at least two positively charged amino acid residues.

4. The peptide according to claim 1, having at least 50% of the positively charged amino acid residues.

5. The peptide according to claim 1, wherein the positively charged amino acid is arginine, histidine, lysine, ornithine, diaminobutyric acid (Dab), diaminopropionic acid (Dap), 2-amino-4-guanidobutyric acid (Agb), 2-amino-3-guanidopropionic acid (Agp), or 2-amino-6-guanidohexanoic acid (Agh).

6. The peptide according to claim 1, wherein the hydrophobic amino acid is isoleucine, leucine, alanine, methionine, phenylalanine, tryptophan, tyrosine or valine.

7. The peptide according to claim 1, wherein the amino acid residues are D-form amino acids.

8. The peptide according to claim 1, wherein the amino acid residues include at least one non-proteinogenic amino acid selected from the group consisting of ornithine, diaminobutyric acid (Dab), diaminopropionic acid (Dap), 2-amino-4-guanidobutyric acid (Agb), 2-amino-3-guanidopropionic acid (Agp), 2-amino-6-guanidohexanoic acid (Agh) and any combination thereof.

9. The peptide according to claim 1, further comprising at least one modification group.

10. The peptide according to claim 9, wherein the at least one modification group is an N-terminal modification group, and the N-terminal modification group is a linear or branched acyl group selected from the group consisting of a formyl group, an acetyl group, a propionyl group, a butyryl group, a pentanoyl group, a hexanoyl group, a heptanoyl group, an octanoyl group, a nonanoyl group, a decanoyl group, a dodecanoyl group, a myristyl group, a palmitoyl group, and a stearyl group.

11. A composition comprising the peptide of claim 1, wherein the composition is a preservative, an antibiotic, or a disinfectant.

12-13. (canceled)

14. A method of preventing or treating a plant disease comprising applying the peptide of claim 1 to a plant in need thereof.

15. The method of claim 14, wherein the plant disease is caused by phytopathogenic bacteria or fungus.

16. The method of claim 15, wherein the phytopathogenic bacteria is Xanthomonas euvesicatoria, Xanthomonas campestris pv. Campestris, Xanthomonas oryzae pv. oryzae, Agrobacterium tumefaciens, Pectobacterium carotovorum subsp. carotovorum, Erwinia chrysanthemi, Pseudomonas syringae pv. tomato, Pseudomonas syringae pv. syringae, or Ralstonia solanacearum.

17. The method of claim 15, wherein the phytopathogenic fungus is Colletotrichum gloeosporioides or Botrytis cinereal.

18. A method of preventing or treating an infection in an animal comprising administering the peptide of claim 1 to the animal in need thereof.

19. The method of claim 18, wherein the infection is caused by at least one of gram-negative bacteria, gram-positive bacteria, fungus and yeast.

20. The method of claim 18, wherein the infection is caused by Escherichia coli, Acinetobacter baumannii, Klebsiella pneumonia, Pseudomonas aeruginosa, Staphylococcus aureus, Staphylococcus epidermidis, Salmonella typhimurium, Candida albicans, Cryptococcus neoformans, Cryptococcus deuterogattii, or Cryptococcus gattii.


SEQUENCE LISTING (29 sequences)

SEQ ID NO: 1 - protein, 18 aa, Artificial Sequence
  artificially synthesized peptide
WKLLKLLLKLLKLLLKLL

SEQ ID NO: 2 - protein, 18 aa, Artificial Sequence
  an artificially synthesized peptide
WKLLKKLLKLLKKLLKLL

SEQ ID NO: 3 - protein, 18 aa, Artificial Sequence
  an artificially synthesized peptide
WKLLKKLLKKLKKLLKKL

SEQ ID NO: 4 - protein, 18 aa, Artificial Sequence
  an artificially synthesized peptide
WKKLKKLLKKLKKLLKKL

SEQ ID NO: 5 - protein, 18 aa, Artificial Sequence
  an artificially synthesized peptide
WKLKLKLKLKLKLKLKLK

SEQ ID NO: 6 - protein, 14 aa, Artificial Sequence
  an artificially synthesized peptide
WKKLKKLLKKLKKL

SEQ ID NO: 7 - protein, 11 aa, Artificial Sequence
  an artificially synthesized peptide
WKKLKKLLKKL

SEQ ID NO: 8 - protein, 14 aa, Artificial Sequence
  an artificially synthesized peptide
  LIPID (1)..(1): MYRISTATE
WKKLKKLLKKLKKL

SEQ ID NO: 9 - protein, 11 aa, Artificial Sequence
  an artificially synthesized peptide
  LIPID (1)..(1): MYRISTATE
WKKLKKLLKKL

SEQ ID NO: 10 - protein, 14 aa, Artificial Sequence
  an artificially synthesized peptide
  LIPID (1)..(1): PALMITATE
WKKLKKLLKKLKKL

SEQ ID NO: 11 - protein, 14 aa, Artificial Sequence
  an artificially synthesized peptide
  LIPID (1)..(1): Stearate
WKKLKKLLKKLKKL

SEQ ID NO: 12 - protein, 14 aa, Artificial Sequence
  an artificially synthesized peptide
WKKIKKIIKKIKKI

SEQ ID NO: 13 - protein, 14 aa, Artificial Sequence
  an artificially synthesized peptide
WKKVKKVVKKVKKV

SEQ ID NO: 14 - protein, 14 aa, Artificial Sequence
  an artificially synthesized peptide
  MISC_FEATURE (2)..(14): d form amino acids
WKKLKKLLKKLKKL

SEQ ID NO: 15 - protein, 11 aa, Artificial Sequence
  an artificially synthesized peptide
  MISC_FEATURE (2)..(11): d-form amino acids
WKKLKKLLKKL

SEQ ID NO: 16 - protein, 14 aa, Artificial Sequence
  an artificially synthesized peptide
WRRLRRLLRRLRRL

SEQ ID NO: 17 - protein, 14 aa, Artificial Sequence
  an artificially synthesized peptide
  MOD_RES (2)..(3): Orn
  MOD_RES (5)..(6): Orn
  MOD_RES (9)..(10): Orn
  MOD_RES (12)..(13): Orn
WXXLXXLLXXLXXL

SEQ ID NO: 18 - protein, 14 aa, Artificial Sequence
  an artificially synthesized peptide
  LIPID (1)..(1): MYRISTATE
WKKVKKVVKKVKKV

SEQ ID NO: 19 - protein, 14 aa, Artificial Sequence
  an artificially synthesized peptide
  LIPID (1)..(1): MYRISTATE
WKKIKKIIKKIKKI

SEQ ID NO: 20 - protein, 11 aa, Artificial Sequence
  an artificially synthesized peptide
  LIPID (1)..(1): MYRISTATE
WKKVKKLVKKL

SEQ ID NO: 21 - protein, 11 aa, Artificial Sequence
  an artificially synthesized peptide
  LIPID (1)..(1): octanoate
WKKLKKLLKKL

SEQ ID NO: 22 - protein, 11 aa, Artificial Sequence
  an artificially synthesized peptide
  LIPID (1)..(1): hexanoate
WKKLKKLLKKL

SEQ ID NO: 23 - protein, 11 aa, Artificial Sequence
  an artificially synthesized peptide
  LIPID (1)..(1): butyrate
WKKLKKLLKKL

SEQ ID NO: 24 - protein, 11 aa, Artificial Sequence
  an artificially synthesized peptide
WKKLHKLLKKL

SEQ ID NO: 25 - protein, 11 aa, Artificial Sequence
  an artificially synthesized peptide
WKKLRKLLKKL

SEQ ID NO: 26 - protein, 11 aa, Artificial Sequence
  an artificially synthesized peptide
WKKLKKALKKL

SEQ ID NO: 27 - protein, 11 aa, Artificial Sequence
  an artificially synthesized peptide
WKKLKKFLKKL

SEQ ID NO: 28 - protein, 11 aa, Artificial Sequence
  an artificially synthesized peptide
WKKLKKILKKL

SEQ ID NO: 29 - protein, 11 aa, Artificial Sequence
  an artificially synthesized peptide
WKKLKKMLKKL